(19) United States
(12) Patent Application Publication
(10) Pub. No.: US 2026/0273190 A1
(43) Pub. Date: Sep. 17, 2026

(54) INJECTION DEVICE

(71) Applicant: SANOFI, Paris (FR)

(72) Inventor: Stefan ALT, Frankfurt am Main (DE)

(21) Appl. No.: 19/469,248

(22) PCT Filed: Mar. 22, 2024
(86) PCT No.: PCT/EP2024/057758

§ 371 Date: Sep. 25, 2025

(30) Foreign Application Priority Data
Mar. 28, 2023 (EP) 23315064.8

(51) Int. Cl. A61M 5/20

(52) U.S. Cl. CPC A61M 5/20; A61M 2005/2006; A61M 2202/0007; A61M 2205/0216; A61M 2205/0233; A61M 2205/3368; A61M 2205/583

(57) ABSTRACT

An injection device for delivering a dose of a medicament includes a housing having an exterior surface and defining a container-receiving space configured to accommodate a medicament container. A thermal conductor is attached to the housing or formed integrally with the housing. The thermal conductor has an interior contact surface that adjoins the container-receiving space or extends into the container-receiving space to make direct mechanical contact with the medicament container when the container is positioned within the space, providing a thermal pathway between the container and the housing.

### CROSS-REFERENCE TO RELATED APPLICATIONS

The present application is the national stage entry of International Patent Application No. PCT/EP2024/057758, filed on Mar. 22, 2024, and claims priority to European Application No. EP23315064.8, filed on Mar. 28, 2023, the disclosures of which are incorporated herein by reference.

### TECHNICAL FIELD

The present disclosure relates to the field of injection devices, in particular to the field of pen-type injectors for injecting one or multiple doses of a medicament. In another aspect the disclosure relates to a method of measuring a temperature of a medicament container or of a respective medicament arranged inside an injection device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of equal or variable size. Moreover, a dose setting as well as a dose dispensing procedure must be easy to operate and has to be unambiguous.

A patient suffering from a particular disease may require a certain amount of a medicament to either be injected via a pen-type injection syringe or by an auto-injector.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

Certain injectable medicament or drugs need to be stored at comparatively low temperature until use, but should not be injected at this storage temperature because the drug effectiveness might be reduced and/or the injection of the rather cold medicament would be more painful. To avoid this, patients are usually advised to remove the injection device with the medicament readily provided therein from a refrigerated storage and let it warm up at room temperature for a certain time. With the true temperature of the fully enclosed medicament unknown, the waiting time has to be chosen with a generous safety margin. But still, it cannot be guaranteed that the medicament has reached the correct temperature at the time of injection.

It would be hence of benefit to actively measure the temperature of the medicament or medicament reservoir inside the injection device. This would allow the patient to shorten the required waiting time as well as to verify that the desired temperature has indeed been reached.

It would be generally desirable to offer a measurement of the temperature of the medicament reservoir in a reliable and easy way and in a cost-efficient manner. This would allow the user to shorten the required waiting time as well as to verify that the medicament has reached a desired temperature before injection.

With some injection devices, such as auto injectors or other hand-held injection devices, such as pen-type injectors allowing a user to individually set and to dispense a dose of a medicament the medicament, the container is readily assembled inside the housing of the injection device. Typically, the medicament container, e.g. in form of a barrel or syringe filled with a liquid medicament, only has a rather limited mechanical contact with a housing of the injection device. It is hence not directly accessible to conduct a temperature measurement from outside the injection device.

It is hence further desirable to enable a temperature measurement of a medicament and/or of a medicament container from outside an injection device while the medicament or medicament container is located inside a housing of the injection device.

### SUMMARY

In one aspect there is proposed an injection device for injecting a dose of a medicament. The injection device comprises a housing with an outside surface. The housing comprises and/or confines a container receiving space. The container receiving space is sized to accommodate a medicament container, wherein the medicament container contains the injectable medicament. The injection device further comprises a thermal conductor attached to the housing or integrated into the housing. The thermal conductor comprises an inside contact surface adjoining the container receiving space or extending into the container receiving face to get in direct mechanical contact with the medicament container when the medicament container is arranged inside the housing and/or occupies the container receiving space.

Typically, the thermal conductor is a dedicated thermal conducting element or thermal conducting member configured to provide a thermal bridge between the medicament container and the housing of the injection device. In this way, at least a portion of the housing, typically that portion, which is in mechanical contact with the thermal conductor, can be thermally coupled with the medicament container located or stored inside the housing. By providing a thermal bridge there can be provided an increased thermal conductivity between an outside surface of the housing and the medicament container and hence between the medicament located inside the medicament container and the outside surface of the housing. In this way, a thermal conductivity between an outside surface of the housing and the medicament located inside the medicament container with the medicament container located or arranged inside the housing can be improved.

This enables to directly measure a temperature at a dedicated portion on the outside surface of the housing, which dedicated portion is in thermal contact with the medicament container and hence with the medicament via the thermal conductor and hence via the respective thermal bridge. Consequently, the dedicated portion of the housing, i.e. a portion of the outside surface of the housing that is in thermal contact with the thermal conductor, allows and supports a rather precise and direct temperature measurement of the medicament container from outside the housing of the injection device. This temperature measurement is indicative of the temperature of the medicament located inside the medicament container.

On the other hand the thermal conductor between the housing and the container receiving space is of particular benefit to improve a transfer of thermal energy from the outside environment of the injection device towards and into the medicament container. Accordingly, and by way of the thermal conductor a time interval required until the medicament inside the medicament container has reached a predefined temperature when the environmental temperature of the injection device is subject to an abrupt change, may significantly decrease. Due to the increased thermal conductivity between the medicament container or medicament and the outside surface of the housing an injection device stored over a comparatively long time in a refrigerated area may heat up substantially faster when transferred into an environment at room temperature compared to an injection device being void of such a thermal conductor.

With a further example of the thermal conductor being attached to the housing or being integrated into the housing and being further configured to get in direct mechanical contact with the medicament container there can be provided an increased and improved support with regard to a mechanical fastening or fixing of the medicament container inside the housing of the injection device. Also here, the thermal conductor provides a twofold function. It enhances the thermal conductivity between the medicament and the outside surface of the housing. At the same time, the thermal conductor provides a mechanically stable mount or support for the medicament container inside the injection device.

With some examples the thermal conductor is provided as a separate piece or member that is attachable or fastenable to the housing of the injection device. With other examples the thermal conductor is readily integrated into the housing. With some examples the thermal conductor may be injection molded into the housing of the injection device. Typically, the housing of the injection device comprises a plastic material or is made of a plastic material. The thermal conductor may be implemented as a separate part made of a material that exhibits a thermal conductivity being larger than the thermal conductivity of the base material of the housing.

According to a further example the thermal conductor comprises a heat conducting material with a thermal conductivity being larger than 0.5 W/(mK), larger than 0.5 W/(mK), larger than 1 W/(mK), larger than 5 W/(mK), larger than 10 W/(mK), larger than 100 W/(mK), larger than 200 W/(mK), larger than 300 W/(mK), larger than 350 W/(mK), or larger than 400 W/(mK).

With some examples the thermal conductivity is larger than 5 W/(mK)and smaller than 420 W/(mK). With some examples the thermal conductivity of the material of the thermal conductor is larger than 100 W/(mK) and smaller than 420 W/(mK). Typically, the heat conducting material of the thermal conductor comprises or exhibits a thermal conductivity that is larger than the thermal conductivity of any of the following plastic materials: polyethylene, polypropylene, polyoxymethylene.

According to a further example the thermal conductor comprises at least one of a metal, a plastic material with a heat conducting dopant, a polymer with a heat conducting dopant, an elastomeric material with a heat conducting dopant, a glass with a heat conducting dopant, a coiled metal wire, a metal ribbon, which may be also coiled, and a material with a heat conductive coating.

With some examples and when the thermal conductor is made of a metallic material it may be unitarily formed. It may consist of a metal interior and may be implemented as a single-pieced or unitary component that is either manually attached to the housing of the injection device or which may be integrated into the housing of the injection device, e.g. by insert molding.

With other examples, e.g. when the thermal conductor comprises a plastic, a polymeric or an elastomeric material with a heat conducting dopant it may be implemented as an injection molded component. It may be provided as a separate injection molded component that is assembled or attached to the housing by a mechanical assembly process. With other examples the respective thermal conductor may be integrated into the housing or may be fastened to the housing by way of a two-or more component injection molding process.

With other examples and when integrated into the housing it is also conceivable that the housing of the injection device is made of a heat conducting material with a thermal conductivity larger than 0.5 W/(mK), larger than 0.5 W/(mK), larger than 1 W/(mK), larger than 5 W/(mK), larger than 10 W/(mK), larger than 100 W/(mK), larger than 200 W/(mK), larger than 300 W/(mK), larger than 350 W/(mK), or larger than 400 W/(mK). Then, the housing itself may provide a respective thermal conductor.

With some examples and when making use of a heat conducting dopant or heat conductive coating the dopant material typically exhibits a comparatively large thermal conductivity, e.g. which is larger than 5 W/(mK), larger than 10 W/(mK), larger than 100 W/(mK), larger than 200 W/(mK), larger than 300 W/(mK), larger than 350 W/(mK), or larger than 400 W/(mK). Here, particles of carbon, graphene or metal may serve or may be used as respective heat conducting dopants, e.g. inside the bulk of a plastic material, a polymer material or an elastomeric material or in a respective coating.

In this way and by making use of a respective heat conducting dopant, the thermal conductivity of respective dopant embedding materials, such as various plastic materials, polymeric materials or elastomeric materials, can be substantially increased in order to serve as a thermal conductor of the present injection device.

According to a further example the housing of the injection device defines a longitudinal direction. The thermal conductor is compressible or deformable with regards to a radial direction extending substantially perpendicular to the longitudinal direction. With some examples the housing is of rather elongated shape. It may extend along an axial direction. This way, the thermal conductor may be then compressible or deformable with regards to the radial direction extending substantially perpendicular to the axial direction.

The compressibility or deformation capability of the thermal conductor may be of particular benefit to provide a geometric tolerance compensating arrangement of the medicament container and the thermal conductor inside the housing of the injection device.

With some examples, the geometric size of the medicament container may be subject to certain tolerances. By providing a thermal conductor being compressible or deformable at least to a certain, e.g. in the region of expected tolerances of the housing and/or medicament container, such geometric tolerances can be effectively compensated, thereby still providing a comprehensive and hence sufficient mechanical and thermally conducting mechanical contact between the medicament container and the thermal conductor.

The same may also apply to the housing, which may be also subject to certain geometric tolerances. In a likewise manner, and by way of a compressible or deformable thermal conductor, respective tolerances of the housing can be effectively compensated. In this way and by providing a compressible or deformable thermal conductor, mechanical forces applied onto the medicament container when arranging the medicament container inside the housing may not exceed a predefined allowable maximum. Therefore, and when making use of a compressible or deformable thermal conductor, optional external forces applied onto the medicament container, e.g. in the course of a final assembly of the medicament container into the injection device, can be reduced to a well-defined minimum, which is beneficial for the container integrity. Moreover, a compressible or deformable thermal conductor may serve to provide a well-defined mechanical fixing of the medicament container inside the housing of the injection device.

According to a further example the housing of the injection device comprises a sidewall and the thermal conductor is arranged between the sidewall and the medicament container or the thermal conductor is arranged between the sidewall and the container receiving space, which is enclosed or confined by the sidewall. Typically, the thermal conductor may be sized to fit into a gap between the medicament container and an inside of the sidewall of the housing. In this way, the thermal conductor provides an effective thermal bridge between the medicament container and the housing. The thermal conductor may at least partially bridge an air gap between an outside container surface and the inside of the sidewall of the housing.

The longitudinal extent of the thermal bridge may be as large as the respective longitudinal extend of the medicament container or container receiving space. With some examples, almost the entirety of e.g. a longitudinal extension of a barrel of the medicament container may be in direct mechanical contact with an at least partially surrounding thermal conductor. In this way, a thermal conductivity between the outside surface of the medicament container and the inside of the sidewall of the housing can be increased to a maximum. This is beneficial for measuring an actual or temperature of the medicament container and hence of the medicament contained therein, as well as to increase a transfer of thermal energy from outside the housing of the injection device towards and into the medicament container.

In this way, the time interval until the medicament has reached a predefined temperature e.g. when taken out of a refrigerated area can be reduced. A waiting time for the patient to conduct an injection can be shortened respectively.

With further examples the thermal conductor comprises a longitudinal extent that is shorter than the longitudinal extend of the medicament container. With some examples the longitudinal extent of the thermal conductor is at least 50% of the longitudinal extent of the medicament container or the container receiving space. With other examples the longitudinal extent of the thermal conductor is at least 30% of the longitudinal extension of the medicament container or the container receiving space. With further examples the longitudinal extent of the thermal conductor is at least 20% of the longitudinal extension of the container receiving space or medicament container.

It is further conceivable that the injection device comprises numerous, hence at least two, three or even more thermal conductors all of which being in thermal contact with the medicament container and with the housing of the injection device. When making use of a number of thermal conductors respective thermal conductors may be arranged longitudinally and/or circumferentially offset from each other, e.g. with regard to a tubular or cylindrical geometry of the medicament container.

Making use of e.g. a first and a second thermal conductor may be beneficial to improve a mechanical fastening of the medicament container inside the housing of the injection device. Here, a first thermal conductor may be arranged close or adjacent to a distal end or a first longitudinal end of e.g. a tubular shaped section of the medicament container and a second thermal conductor may be arranged near or at a proximal and hence a second longitudinal end of the shaped section of the medicament container.

With some examples the thermal conductor may completely or at least partially enclose an outer circumference of the medicament container. With a tubularly shaped medicament container the thermal conductor may comprise an annular shaped body or annular shaped structure having an inside diameter that matches with an outside diameter of the tubular barrel of the medicament container.

With a further example the sidewall of the housing comprises a recess on an inside. The thermal conductor is arranged in the recess on the inside of the sidewall of the housing. Such an arrangement is beneficial in at least two aspects. First of all, the recess on the inside of the sidewall of the housing leads to a reduced thickness of the housing in the region of the recess. In this way, the thermal coupling between the thermal conductor and the outside surface of the housing can be improved presumed that the thermal conductivity of the material of the sidewall of the housing is smaller than the thermal conductivity of the thermal conductor. Second, by way of the recess on the inside of the sidewall of the housing, the thermal conductor can be precisely and easily fixed inside the housing. With some examples the geometry of the recess is complementary shaped to the geometry of the thermal conductor. This allows for a form fitting fixing of the thermal conductor inside the recess on the inside of the sidewall of the housing of the injection device.

The recess on the inside of the sidewall is hence of particular use for a mechanical fastening or fixing of the thermal conductor inside the housing. Moreover, and when provided with a respective recess a housing of an injection device could be also easily retrofitted with a thermal conductor to improve the thermal conductivity between the medicament container and the outside surface of the housing. This way, one and the same housing type could be used for the production and assembly of injection devices with or without such thermal conductors. Housings of injections devices intended for use with a particular medicament could be then individually configured and provided with a respective thermal conductor.

According to a further example at least a portion of the thermal conductor protrudes inwardly from the inside of the sidewall of the housing of the injection device. In this way, there can be provided a well-defined mechanical contact between the inside contact surface of the thermal conductor and an outside surface of the medicament container when the medicament container is assembled inside the housing. Furthermore, the inwardly protruding thermal conductor may also mechanically stabilize and fix the medicament container inside the housing of the injection device. In this way, the inwardly protruding thermal conductor provides a twofold function. It improves the thermal conductivity between the outside surface and the medicament container and further helps to fix the medicament container inside the housing of the injection device.

According to a further example the recess as provided on the inside of the sidewall of the housing of the injection device comprises a through opening extending all through the sidewall of the housing. Here, the thermal conductor may effectively fill the opening or may extend through the through opening. In this way, the thermal conductor may participate or may contribute to the outside surface of the housing. By reaching through or by filling the through opening and hence by extending through the sidewall of the housing the thermal conductor may become directly accessible from outside the housing of the injection device. Such a configuration is of particular benefit to measure the temperature of the thermal conductor and hence the temperature of the medicament container, which is in thermal contact with the thermal conductor.

Moreover, a thermal conductor contributing to the outside surface of the housing of the injection device helps to improve and to accelerate a transfer of thermal energy from the environment of the injection device towards and into the medicament container or medicament.

According to a further example the thermal conductor comprises an outside contact surface that flushes with the outside surface of the housing. Here, the thermal conductor, which may extend through the through opening of the housing and which may optionally entirely fill the through opening, contributes to the outside surface of the housing in a rather elegant way. By not protruding from an outside surface of the housing and by flushing with the outside surface of the housing the thermal conductor has no influence on the handling of the injection device, to which a user may be used to. Moreover the flush arrangement of the thermal conductor on or in the outside surface of the housing allows and supports an attachment of a temperature measurement device, such as a temperature measuring tag or electronic label on the outside surfacer of the housing.

Moreover, with the thermal conductor extending through the sidewall of the housing a user may even manually detect the temperature of the medicament container being in thermal contact with the thermal conductor, e.g. by bringing a temperature sensitive body portion, such as a finger or a cheek in direct mechanical contact with the outside contact surface of the thermal conductor.

If the outside contact surface of the thermal conductor can be manually sensed to be rather cool the patient or user of the injection device receives a direct indication that the medicament container and hence the medicament located therein is still at a temperature level that would be unfavorable when injected.

The thermal conductor flushing with the outside surface of the housing may also enhance or improve the visual design of the injection device. The thermal conductor may distinguish in color and/or with regard to its haptic structure or texture from the residual outside surface of the housing. In this way, the presence of the thermal conductor with the injection device is immediately apparent to a skilled user. Moreover, the visual appearance of the thermal conductor on or in the outside surface of the housing of the injection device provides a rather direct approach to conduct a temperature measurement, e.g. by making use of an auxiliary temperature measuring device.

According to a further example the inside contact surface of the thermal conductor is complementary shaped to an outside surface of the medicament container. With some examples the medicament container comprises a tubular-shaped barrel. Then, the inside contact surface of the thermal conductor may be also tubularly-shaped or may comprise a tubular-shaped section that is configured to establish a comparatively large surface contact with the outside surface of the medicament container.

With a tubularly-or cylindrically-shaped medicament container the inside contact surface may comprise a respective tubular or semi-tubular shaped structure so as to maximize a mutual contact surface between the thermal conductor and the outside surface of the medicament container.

With a further example the injection device comprises a medicament container fixed or assembled inside the housing. The medicament container is in direct mechanical and thermal contact with the thermal conductor. The medicament container may be a prefilled medicament container. Hence, the medicament container is readily filled with a liquid medicament before it is assembled inside the housing of the injection device.

The injection device may be implemented as a disposable injection device with the medicament container readily assembled therein. Disposable injection devices are intended to be discarded in their entirety after use of the injection device. Use of the injection device may include a single or multiple doses of the medicament. With some examples the injection device is implemented as a fixed dose injection device. It may be implemented to inject one or multiple doses of equal sizes. With other examples the injection device is configured to individually set a dose or several doses of different dose size and to subsequently inject the respective dose.

With some examples the injection device is implemented as a reusable injection device. Here, the housing may be configured to become disassembled or to provide an opening allowing to replace the medicament container.

With some examples the medicament container is permanently fixed inside the housing of the injection device. With other examples the medicament container may be movably disposed inside the injection device. With some examples the injection device is implemented as a so-called auto-injector, where the injection device provides a movement of an injection needle relative to the housing of the injection device to automatically pierce the skin of the patient and to administer the dose of the medicament subsequently. With some examples the medicament container may comprise a needle permanently and non-removably fixed to an outlet of the medicament container. With other examples the injection device comprises a needle assembly detachably connectable to the injection device and/or to the medicament container.

In either way, a proximal end of the injection needle is located inside or can be inserted in an outlet end of the medicament container to dispense or to inject a dose of the medicament by moving a stopper of the medicament container in distal direction, i.e. towards the outlet end of the medicament container. A displacement of a stopper of the medicament container is typically induced by a drive mechanism of the injection device, which may comprise a piston rod to get in mechanical thrust exerting contact with the stopper of the medicament container. The stopper of the medicament container typically seals the interior of a tubular-shaped barrel of the container towards the proximal end, which is opposite the dispensing outlet of the medicament container.

According to a further example of the injection device the housing and/or the injection device comprises a marking on the outside surface of the housing. The position of the marking overlaps with a position of the thermal conductor inside the housing. Hence, a portion of the housing visibly or haptically marked by the marking indicates a dedicated portion of the housing that is particularly suitable to conduct a temperature measurement being indicative of the temperature of the medicament container located inside the housing.

The dedicated portion of the housing, which is indicated by the marking, is in mechanical and/or thermal contact with the thermal conductor and thus contributes to the mechanical bridge between the outside surface of the housing and the medicament container.

According to a further example the injection device comprises a temperature sensor fastened to an outside of the housing and being in thermal contact with the thermal conductor. Typically, the temperature sensor may be arranged in an overlapping configuration with the marking optionally provided on the outside surface of the housing. With some examples the marking on the outside surface of the housing may be provided by the thermal conductor extending through the through opening of the sidewall of the housing as described above. Here, the thermal conductor itself provides a visible marking to provide a rather precise temperature measurement of the thermal conductor and hence of the medicament container.

With some examples the temperature sensor is part of a machine-readable label configured for attachment to the outside surface of the injection device. The machine-readable label may comprise a substrate, e.g. a flexible substrate configured for wrapping around the outside surface of the housing and/or for fastening to an outside surface of the housing, which may be of elongated tubular shape. The temperature sensor may be part of an auxiliary device that is attachable or fastenable to the housing of the injection device. The auxiliary device may comprise a respective temperature sensor. The auxiliary device may be implemented as one of a machine-readable label or as an injection monitoring device, which is operable to record and/or or to electronically store a single or repeated use of the injection device. With some examples the auxiliary device may be operable to quantitatively measure, to detect and/or to record or to store a size of a dose and a time or date at which the respective dose has been injected by the injection device.

With some examples the machine-readable label comprises an electronic circuit. The electronic circuit may be implemented as an integrated circuit. It may be printed on a substrate of the machine-readable label. The electronic circuit may comprise a processor or a controller and a temperature sensor connected to the processor or controller. Furthermore, the machine-readable label may comprise an antenna or a communication module connected to the processor and operable to communicate with an external electronic device. With some examples, the electronic circuit is a passive electronic circuit operable to withdraw or to harvest electric energy or electric power from a radiation source, e.g. of the external electronic device. The communication interface may comprise a wireless antenna, e.g. operable to transmit RF signals. With some examples the machine-readable label may be implemented as a RFID tag, as a NFC tag or as a Bluetooth low energy device tag by way of which a temperature measurement can be conducted.

With some examples the machine-readable label is a passive NFC or RFID label without an own electrical source of energy. It may be driven by an external electromagnetic field, e.g. provided by a reading device, such as the external electronic device. Typical external electronic devices to cooperate or to communicate with the machine-readable label may be implemented as a smart watch, as a smart phone, as a tablet computer or any other computing or smart device.

Energy for operating the temperature measurement may be harvested from an electromagnetic RF field as provided by the reading device. Such energy is typically harvested by the antenna of the machine-readable label. Once the machine-readable label has received sufficient electrical energy from the reading device it becomes operable to conduct a temperature measurement through the temperature sensor being connected to the processor of the machine-readable label. A temperature signal as provided by the temperature sensor may be suitably processed by the processor of the machine-readable label and may be wirelessly transmitted to the external electronic device or reader, which in turn is operable to illustrate or to communicate the measured temperature to a user of the respective devices.

Machine-readable label providing a temperature measurement may be commercially available on the market. They may be implemented as temperature measuring NFC tag with an integrated temperature sensor. The temperature measurement can be performed in comparatively short intervals upon activation of the NFC tag, namely when activated by the external reading device.

The machine-readable label may be provided with an adhesive layer, which allows to fix the label to the dedicated portion on the outside surface of the housing, which is in thermal contact with the thermal conductor. With some examples the machine-readable label is a flexible label. It may be wrapped around and e.g. adhesively fixed to the injection device.

The machine-readable and temperature sensing label or any other auxiliary device may be readily fastened to the injection device upon delivery to a patient or end consumer. With other examples the injection device and the auxiliary temperature measuring device may be provided separately to the patient or user of the respective devices. Then, the injection device is typically provided with a marking on the outside surface indicating a portion, where the temperature measurement should be conducted. The reading device, either in form of an auxiliary dose measurement device or in form of a machine-readable label, may be then provided with a complementary shaped indicator, which when overlapping with the marking upon attachment of the auxiliary device or machine-readable label on the housing of the injection device provides that the temperature sensors suitably overlaps with the thermal conductor.

According to another aspect the present disclosure also relates to a method of measuring a temperature of a medicament located inside a medicament container, which medicament container is arranged inside a housing of an injection device as described above.

The method comprises the step of indirectly measuring the temperature of the medicament through measuring of a temperature of the thermal conductor by making use of the temperature sensor. Here, the thermal conductor provides a thermal bridge between the medicament container and hence the medicament located inside the housing of the injection device and the temperature sensor, which has only access to the outside surface of the housing of the injection device. The method of measuring the temperature of the medicament is typically conducted with an injection device as described above. Insofar, all features, effects and benefits as described above in connection with the injection device equally apply to the method of measuring the temperature of the medicament.

According to another aspect the present disclosure also relates to a method of heating or cooling a medicament located inside a medicament container, wherein the medicament container is arranged inside a housing of an injection device as described above. Here, the method comprises the steps of moving the injection device out of a first area into a second area, wherein a temperature of the first area differs from a temperature of the second area. Thereafter, thermal energy is exchanges or transferred between the second area and the medicament container via the thermal conductor of the injection device.

The method is suitable for a comparatively fast heating or cooling of the medicament container when arranged inside the injection device and when the housing and/or the assembly of the medicament container inside the housing is of a comparatively low thermal conductivity. With some examples the housing of the injection device is made of a plastic material comprising a comparatively low thermal conductivity, e.g. lower than 0.5 W/(mK). In addition, there may be provided air gaps between a sidewall of the medicament container and an inside of a sidewall of the housing, which air gaps also exhibit a comparatively poor thermal conductivity. By way of the thermal conductor there can be provided a thermal bridge between an outside container surface and the outside surface of the housing of the injection device.

With some examples the temperature of the first area is smaller than a temperature of the second area. This is for instance the case when the injection device is taken out of a refrigerated area and is subsequently stored at room temperature before an injection procedure will be conducted. It is then that the thermal conductor provides a transfer of heat or thermal energy from the second area into the medicament container. Vice versa and when the injection device returns into a refrigerated area after use, the temperature of the first area is larger than a temperature of the second area. Accordingly, respective cooling thermal energy is transferred from the second area into the medicament container. Accordingly, a cooling effect for the medicament container after returning the injection device into the refrigerated area can be accelerated.

In another aspect the present disclosure also relates to a thermal conductor for an injection device. The injection device comprises a housing confining a container receiving space, which is sized to accommodate a medicament container filled with a medicament. The thermal conductor comprises an inside contact surface to adjoin or to extend into the container receiving space and to get in direct mechanical contact with the medicament container when the medicament container is arranged in the container receiving space.

Here, the thermal conductor may be provided as a separate part without being assembled or integrated in the injection device as described above. Apart from that, the thermal conductor may be substantially identical to the thermal conductor of the injection device as described above. Insofar, any features, effects and benefits as described above in connection with the injection device equipped with the thermal conductor equally apply to the thermal conductor as such and as applicable.

According to a further example the thermal conductor comprises a heat conducting material with a thermal conductivity being larger than 0.5 W/(mK), larger than 0.5 W/(mK), larger than 1 W/(mK), larger than 5 W/(mK), larger than 10 W/(mK), larger than 100 W/(mK), larger than 200 W/(mK), larger than 300 W/(mK), larger than 350 W/(mK), or larger than 400 W/(mK).

According to a further example the thermal conductor comprises at least one of a medical, a plastic material your (corresponding to claim 3). Moreover and as described above in connection with the thermal conductor of the injection device also the isolated thermal conductor may be compressible or deformable with regard to a radial direction transfers to a longitudinal direction of the housing.

According to a further example the thermal conductor comprises at least one of a metal, a plastic material with a heat conducting dopant, a polymer with a heat conducting dopant, an elastomeric material with a heat conducting dopant, a glass with a heat conducting dopant, a coiled metal wire, a metal ribbon, which may be also coiled, and a material with a heat conductive coating.

With a further example, the thermal conductor may comprise a cylindrically shaped body, which may be configured to at least partially enclose or to clasp around the medicament container.

With another example, the thermal conductor may comprise a geometric shape and geometry to fit into a recess on an inside of the sidewall of the injection device and/or to fill a through opening provided in a sidewall of the injection device.

According to a further example the thermal conductor comprises an outside contact surface configured to establish a thermal contact with a temperature sensor configured to be arranged to an outside surface of the housing of the injection device.

With a further example the thermal conductor is attachable or fastenable to an outside surface of a sidewall of a barrel of the medicament container. The medicament container may be provided as a medicament cartridge, as a medicament carpule, as a syringe or as a vial.

According to a further example the thermal conductor is attachable to the housing of the injection device or the thermal conductor is integratable into the housing of the injection device.

In another aspect the disclosure further relates to a medicament container comprising a barrel at least partially filled with an injectable medicament. The barrel comprises a sidewall, e.g. of tubular shape. The barrel may comprise a distally located outlet for expelling of the medicament from the interior volume of the barrel and may further comprise a proximal end provided with a movable plunger or stopper for expelling the liquid medicament through the outlet. The outlet may be provided with an injection needle for injecting the medicament into biological tissue. The medicament container further comprises a thermal conductor as described above, which is attached or fastened to an outside surface of the sidewall of the barrel. Insofar, all effects, features and benefits as a described above in connection with the thermal conductor equally apply to the medicament container equipped with such a thermal conductor. Typically, the medicament container and the thermal conductor are configured for assembly inside a housing of an injection device as described above. Insofar, all effects, features and benefits as described above in connection with the injection device equally apply to the medicament container; and vice versa.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(w-carboxyheptadecanoyl)-des(B30) human insulin and B29-N-(w-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF THE DRAWINGS

In the following, examples of injection devices with a thermal conductor for temperature measurement and methods of using the same will be described in greater detail by making reference to the drawings, in which:

FIG. 1 schematically shows an example of an injection device equipped with a medicament container and a thermal conductor,

FIG. 2 is a three-dimensional view of another injection device in combination with a machine-readable electronic label and an external electronic device for temperature measurement,

FIG. 3 schematically illustrates the thermal bridge for a temperature measurement to be conducted with the thermal conductor, an electronic label and the external electronic device,

FIG. 4 schematically illustrates an example of a thermal conductor,

FIG. 5 shows another example of a thermal conductor,

FIG. 6 schematically illustrates one example of an injection device with a thermal conductor inside the housing of the injection device,

FIG. 7 shows the example of FIG. 6 with the medicament container arranged inside the housing,

FIG. 8 schematically illustrates another example of attaching or integrating the thermal conductor in the housing of the injection device,

FIG. 9 shows a further example of an arrangement of a housing and a thermal conductor,

FIG. 10 schematically illustrates a variation of a temperature of the medicament container over time when taken out of a refrigerated area,

FIG. 11 shows a flowchart of a method of measuring a temperature of the medicament,

FIG. 12 is a cross section through an example of a thermal conductor,

FIG. 13 is a cross section through another example of a thermal conductor, and

FIG. 14 shows another example of a thermal conductor.

### DETAILED DESCRIPTION

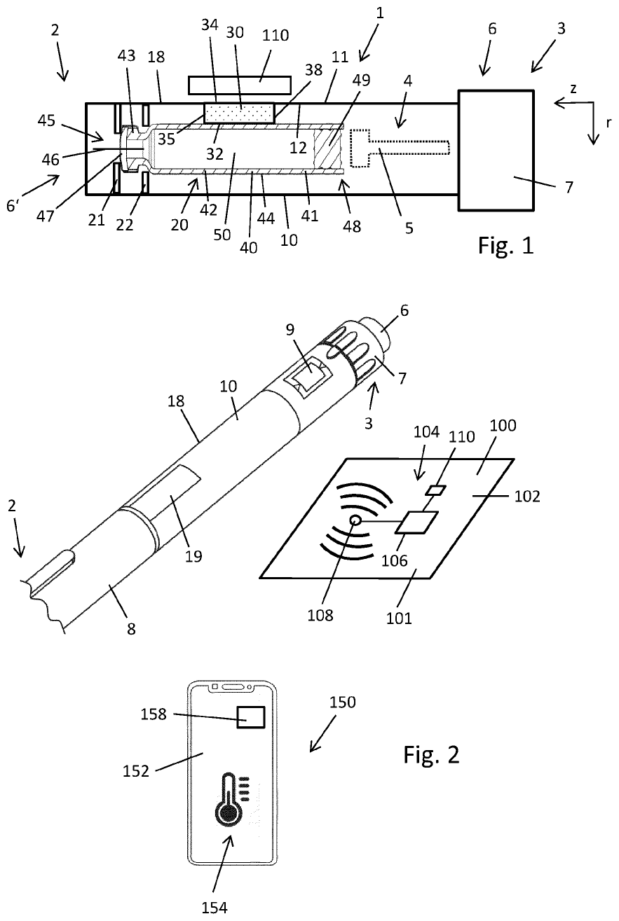

In FIGS. 1 and 2 there are shown examples of an injection device 1, e.g. implemented as a handheld pen-type injector. The injection device 1 comprises a housing 10. Inside the housing 10 there is provided a container receiving space 20, which is indicated in FIG. 6. The container receiving space 20 is typically defined by the outside dimensions or geometry of a medicament container 40, which is configured to be received and to be accommodated inside the housing 10 of the injection device 1. When assembled inside the housing 10 the medicament container 40 completely occupies the container receiving space 20.

The medicament container 40 may comprise a tubular-shaped barrel 42 featuring a tubular-shaped sidewall 41. The medicament container 40 may comprise an outlet 43 at or near a distal end 45 and may further comprise a movable stopper 49 in or near an opposite longitudinal proximal end 48. With some examples the outlet 43, as provided at the distal end 45 of the medicament container 40, comprises a septum 47 pierceable by an injection needle 46. Here, the injection needle 46 may be operable to penetrate the septum 47 to obtain access to the liquid medicament 50 stored inside the medicament container 40.

With some examples the injection needle 46 may be movably or detachably connectable to the outlet 43. Here, the injection needle 46 may belong to or may be part of a needle assembly (not illustrated), which is configured to detachably connect to a distal end of the housing 10, e.g. of a cartridge holder portion of the housing 10 of the injection device 1.

With other examples the injection needle 46 is permanently fixed to the outlet 43. It may reach through the outlet and may be permanently fastened to the barrel 42. It may be embedded in the material of the barrel 42. The barrel 42 typically comprises a pharmaceutically inert material, e.g. a vitreous material, such as glass.

The medicament container 40 as presently illustrated is of tubular and elongated shape. Its distal end 43 may coincide with the outlet 43. Opposite the distal end 45 there is provided the proximal end 48, which is sealed by the movable stopper 49. For expelling a dose of the medicament through the outlet 43 and hence through the injection needle 46 provided at or extending through the outlet 43 the injection device 1 typically comprises a drive mechanism 4 including a piston rod 5, which is displaceable in distal direction and hence towards the outlet 43 of the medicament container 40. The piston rod 5 is configured to engage with or to urge against the stopper 49 in distal direction so as to increase the fluid pressure inside the barrel 42 thereby expelling a well-defined amount or dose of the medicament, the size of which correlates to the displacement of the stopper 49 relative to the sidewall 41 of the medicament container 40.

The housing 10 comprises at least one container support 21, 22 configured to mechanically engage with the medicament container 40. By way of the container support 21, 22 the medicament container 40 can be mechanically fixed inside the housing 10. With the example of FIG. 1 the container support 21 is provided at or near the distal end 45 of the medicament container 40. The optional container support 22 is provided at or near a shoulder portion of the medicament container 40. Both container supports 21, 22 are configured to provide an axial or longitudinal support for the medicament container 40 inside the housing 10 so as to counteract a distally directed dispensing force as applied by the piston rod 5 onto the stopper 49 of the medicament container 40. There may be provided further support structures, e.g. for keeping the medicament container 40 in a dedicated position inside the housing 10 not only with regard to the longitudinal direction (z) but also with regards to a radial direction (r).

The injection device 1 may comprise a dose dial 7 allowing a user to set a dose and/or to arm the drive mechanism 4. Furthermore, there may be provided a trigger 6 by way of which a dose injection procedure can be triggered. With the injection device 1 implemented as an auto-injector the trigger may be provided at the distal end 2 of the injection device, e.g. in form of a needle sleeve or needle shroud. Here, the trigger 6′ may be integrated into a distal end of the housing 10. With other examples the trigger 6 is provided as a button at or near the proximal end 3 of the injection device 1, which is exemplary illustrated in FIG. 2.

The distal end or 2 of the injection device 1 may be provided with a detachable or removable protective cap 8. The cap 8 may be configured to cover and/or to protect at least one of the outlet 43 and the injection needle 46.

Optionally, the injection device 1 is provided with a window 9. Particularly and with variable dose setting devices the window 9 is operable to visibly indicate the size of a medicament dose currently set and to be dispensed.

With the exception of a thermal conductor 30, the details of which will be described in greater detail below, the injection device 1 as shown herein is representative for a large variety and for different types of injection devices, that comprise a housing with a container receiving space to accommodate a medicament container and which are further operable to inject a dose of the medicament provided inside the medicament container into biological tissue.

The injection device 1 as described herein is provided with a thermal conductor 30, which is attached to the housing 10 or which is integrated into the housing 10. The thermal conductor 30 comprises an inside contact surface 32 adjoining the container receiving space 20 or extending into the container receiving space 20 to get in direct mechanical contact with the medicament container 40, when the medicament container 40 is arranged in the container receiving space 20 of the housing 10 of the injection device 1.

The thermal conductor 30 as illustrated in FIG. 1 serves to provide a thermal bridge between an outside container surface 44 of the medicament container 40 and an outside surface 18 of the housing 10 of the injection device 1. Without the thermal conductor 30, there would be provided a rather poor thermal contact or coupling between the outside surface 18 of the housing 10 and the medicament container 40. Therefore, it may be rather difficult to measure a temperature of the medicament 50 or of the medicament container 40 from outside the housing 10. Moreover, a rather poor thermal conductivity between the outside surface 18 of the housing 10 and the medicament container 40 is detrimental to a fast and desirable thermal heating or cooling of the medicament 50.

Generally, a temperature of the injection device 1 and/or of the medicament container 40 can be measured with an auxiliary electronic device, such as an electronic label 100, schematically illustrated in FIG. 2. The electronic label 100 comprises a substrate 101, which may be flexible. The substrate 101 may be implemented as a flexible foil 102 to be wrapped around the outside surface 18 of the housing 10. It may be provided with an adhesive, e.g. with a self-adhesive layer, such that the electronic label 100 can be permanently attached to the outside surface 118 of the housing 10. The electronic label 100 may be implemented as a passive NFC tag or RFID tag. It may comprise an antenna 108, a temperature sensor 110 and a processor 106. An electronic circuit 104, e.g. comprising the temperature sensor 110, the processor 106 and the antenna 108 may be printed on the substrate 101.

When attached to the housing 10 the electronic label 100 may be arranged and fastened or fixed to the housing 10 in such a way that the temperature sensor 110 overlaps with a marking 19 as provided on the outside surface 18, wherein the marking and/or haptically marks or indicates a position of the thermal conductor 30 of the injection device 1. With some examples the marking 19 may be even provided by the thermal conductor 30 itself.

The marking 19 as visible on the outside surface 18 of the housing 10 may coincide with an outside contact surface 34 of the thermal conductor 30. In this way and when suitably attaching the electronic label 100 to the outside surface 18 of the housing 10 the temperature sensor 110 may overlap with the marking 19 and/or with the thermal conductor 30, thereby obtaining a rather direct thermal coupling between the temperature sensor 110 and the medicament container 40 via the thermal bridge, which is provided through the direct mechanical and hence thermal contact between the medicament container 40, the thermal conductor 30 and the temperature sensor 110.

The electronic label 100 may be operable to communicate with an external electronic device 150. The external electronic device 150 may be implemented as a wireless reader operable to exchange electromagnetic signals with the electronic label 100. The external electronic device 150 comprises a communication interface 158 that is configured to exchange signals with the antenna 108 of the electronic label 100.

Typically, the communication interface 158 comprises a NFC reader or RFID reader to provide electromagnetic energy to the passive electronic label 100 in order to wake up and to activate the electronic label 100. Moreover, the communication interface 158 is operable to receive electromagnetic signals from the antenna 108, which electromagnetic signals are indicative of sensor signals from the temperature sensor 110.

The external electronic device 150 is typically operable to process the sensor signals received from the electronic label 100 and is further operable to visually display or to audibly provide information about the temperature as measured by the temperature sensor 110. A respective temperature indication 154 may be provided on a display 152 of the external electronic device 150.

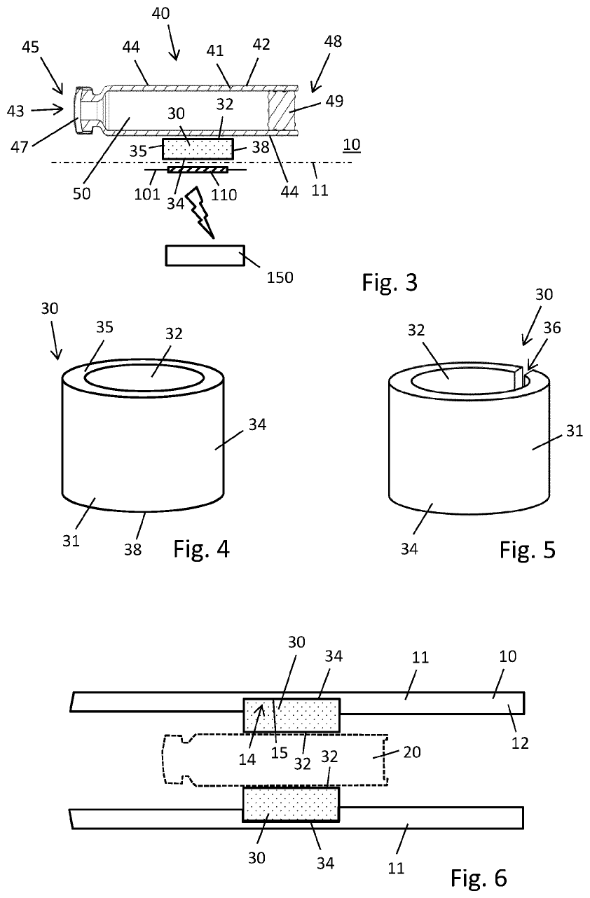

An example of the thermal bridge as provided by the thermal conductor 30 is illustrated in greater detail in FIG. 3. The thermal conductor 30 is arranged inside the housing 10 of the injection device 1. It may be integrated into a sidewall 11 of the housing 10 or may contribute to the sidewall 11 of the housing 10. The thermal conductor 30 comprises an inside contact surface 32, that is complementary shaped to the outside container surface 44. This way, a mutual contact region between the thermal conductor 30 and the sidewall 41 of the medicament container 40 can be maximized so as to improve the thermal conductivity between the sidewall 41 or barrel 42 of the medicament container 40 and the thermal conductor 30.

The thermal conductor 30 exhibits a particular thermal conductivity, which is typically larger than the thermal conductivity of the housing 10. The thermal conductivity of the material of the thermal conductor 30 may be larger than the thermal conductivity of undoped plastic or glass. It may be also larger than the thermal conductivity of undoped and hence conventional plastic materials, such as polyethylene or polypropylene or polyoxymethylene.

The thermal conductor 30 further comprises an outside contact surface 34, which is opposite the inside contact surface 32. The outside contact surface 34 of the thermal conductor 30 may get in direct or indirect thermal contact with the temperature sensor 110 of the electronic label 100. Optionally, there may be provided the substrate 101 and/or a portion of the sidewall 11 of the housing 10 between the outside contact surface 34 and the temperature sensor 110. The temperature sensor 110 is connected to the processor 106. Measurement signals generated or modified by the temperature sensor 110 can be wirelessly transmitted to the external electronic device 150 via the mutual interaction between the antenna 108 and the communication interface 158 of the external electronic device 150.

Typically, the external electronic device 150 is implemented as a smartwatch, as a smart phone or as a tablet computer. It is typically provided with a software application, e.g. an app, configured to translate or to calculate measurement signals received from the electronic label 100 into a visual indication of a temperature on the display 152 of the external electronic device 150.

As further illustrated in FIGS. 3 and 4 the thermal conductor 30 comprises a distal end 35, which in the assembled state inside the housing 10 faces towards the distal end 45 of the medicament container 40. The thermal conductor 30 further comprises a proximal end 38 facing towards the proximal end 48 of the medicament container 40. The longitudinal extent of the thermal conductor 30 may be as large as the longitudinal extent of the respective medicament container. With some examples the longitudinal extent, hence the distance between the distal end 35 and the proximal end 38 of the thermal conductor 30 may be larger than 10%, larger than 20%, larger than 30% or larger than 50% of the longitudinal extent of the medicament container 40. In this way a substantial portion of the outside container surface 44 of the barrel 42 can get in direct mechanical and hence thermal contact with the thermal conductor 30.

Also with regard to a circumferential direction of the medicament container 40, the thermal conductor 30 may clasp or extend around the entire circumference of the medicament container 40. Here, the thermal conductor 30 may comprise an annular closed ring or an annular or tubularly-shaped closed structure. With further examples, the thermal conductor may comprise an size in circumferential direction that is larger than 180° of the tubular shaped circumference of the barrel 42. With some examples, the circumferential extent of thermal conductor 30 is between 90° and 270° of the circumference of the outside container surface 44 of the barrel 42.

In either way and with the thermal conductor 30, a time interval until the medicament container 40 reaches a thermal equilibrium with the environment can be reduced to a minimum after the injection device has been subject to an abrupt change of the temperature of its environment, e.g. when the injection device is taken out of a refrigerated area and is kept or stored at room temperature prior to an injection.

As particularly illustrated in FIG. 4 and with some examples the thermal conductor 30 may comprise a tubular cylindrical sleeve featuring a hollow cylindrical inside contact surface 32 and a corresponding cylindrical outside contact surface 34. The tubular shaped body 31 of the thermal conductor 30 may be made of a metal, such as aluminum, copper or brass, exhibiting a comparatively high thermal conductivity. The thermal conductor 30 as illustrated in FIG. 4 may be made of a solid and hence rigid material. It may be comparatively stiff or rigid.

The thermal conductor 30 may be mounted as a separate part inside the housing 10 of the injection device. Here, the outside contact surface 34 may be in engagement with an inside 12 of the sidewall 11 of the housing 10. With some examples the thermal conductor 30 may be injection molded to or into the injection molded housing 10. It may be insert molded and may provide a thermally conducting insert inside the sidewall structure 11 of the housing 10.

With the example of FIG. 5 the body 31 of the thermal conductor 30 is also of cylindrical shape but comprises a longitudinal slit 36 extending all through the body 31 of the thermal conductor 30. The longitudinal slit 36 provides the thermal conductor 30 with a certain degree of elasticity, compressibility or deformability. Here, and when the thermal conductor 30 is made of a comparatively rigid metal it may be at least deformable to a certain degree, thus allowing to adapt the thermal conductor to inevitable manufacturing or geometric tolerances of both, the housing 10 and the medicament container 40.

With the example of FIG. 12 the thermal conductor 30 comprises a body 31 made of a plastic material 52, which is provided with embedded heat conducting dopant material 53. The dopant may comprise comparatively small sized particles in the submillimeter range, even nanoparticles with a comparatively high thermal conductivity. With some examples, the dopant material may be one of a metal, carbon or graphene.

With the further example of FIG. 13, the thermal conductor 30 comprises a body 31 provided with a heat conductive coating 54. Here, the coating 54 may be provided on the inside contact surface 32 as well as on the outside contact surface 34. With the further example of FIG. 14 the thermal conductor 30 can comprise a body 31 in the form of a coiled ribbon 56 forming one or more windings 58. Here, the body 31 may comprise a comparatively thin sheet of a material, such as a comparatively thin layer of a metal sheet, which can be wrapped around the medicament container 40.

As illustrated, the ribbon 56 comprises an inside winding 58′ and an outside winding 58. The windings 58, 58′ may be provided in a densely packed arrangement, such that opposite and mutually adjacent surfaces of the ribbon 56 and hence of the individual windings 58, 58′ are in a comparatively large surface contact. By way of a ribbon 56 and by way of numerous windings the radial thickness of the thermal conductor 30 can be easily adapted to varying demands. Instead of a ribbon 56 the body 31 of the thermal conductor 30 may comprise a single or several wires wrapped around or wrappable around the medicament container 40.

When preinstalled in the inside of the housing 10, the ribbon or wire structure of the thermal conductor may be attached, fixed or fastened to the inside 12 of the sidewall 11. Alternatively, the ribbon or wire 56 of the thermal conductor 30 may be integrated or embedded e.g. insert molded in the sidewall 11 of the housing 10.

With the examples of FIGS. 6 through 9, variations arrangements of the thermal conductor 30 inside the housing 10 are illustrated. In the illustration, the size of the thermal conductor 30 is increased relative to the medicament container 42 to show the thermal bridge in greater detail.

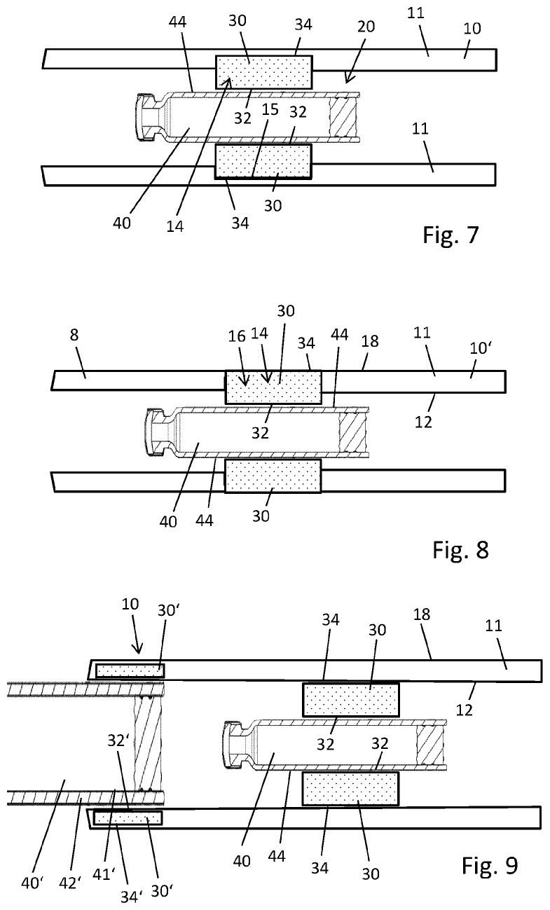

With the examples of FIGS. 6 and 7 the inside 12 of the sidewall 11 of the housing 10 comprises a recess 14. The thermal conductor 30 is arranged in the recess 14. The thermal conductor 30 may completely fill the recess 14 such that the inside contact surface 32 protrudes from the inside 12 of the sidewall 11. This way, the inside contact surface 32 may directly adjoin or may even enter or extend into the container receiving space 20 of the housing 10. With the example of FIG. 7, the container receiving space 20 is completely occupied by the respective medicament container 40. Here, and consequently, the inside contact surface 32 of the thermal conductor 30 is in direct abutment and hence in mechanical as well as thermal contact with the outside container surface 44 of the barrel 42 of the medicament container 40. The geometry of the inside contact surface 32 is complementary shaped to the outside container surface 44.

The inwardly protruding surface portions of the thermal conductor 30 also provide a mechanical stabilization for mounting the medicament container 40 inside the housing 10. Here, the thermal conductor 30 may even provide a substantial portion of the mount or mounting structure for keeping and fixing the medicament container 40 inside the housing 10.

Providing a recess 14 on the inside 12 of the sidewall 11 is further beneficial in that the thickness of the sidewall 11 in the region of the recess 14 is smaller compared to regions of the sidewall 11 offset from the recess 14. Hence, the bottom 15 of the recess 14 is comparatively thin. With examples, wherein the sidewall 11 has a comparatively low thermal conductivity, the reduced thickness of the sidewall 11 in the region of the recess 14 is beneficial to provide an improved thermal conductivity across the sidewall as 11 compared to configurations without such a recess.

With the further example according to FIG. 8, the recess 14 comprises a through opening 16 extending all through the sidewall 11. Here, the thermal conductor 30 extends through the through opening 16. The outside contact surface 34 of the thermal conductor 30 may flush with the outside surface 18 of the housing 10 as illustrated in FIG. 8. This way, there can be provided a direct mechanical as well as thermal contact between the thermal conductor 30 and the temperature sensor 110 or the electronic label 100. The thermal conductivity of the thermal bridge as provided between the medicament container 40 and the outside surface 18 of the housing 10 can be therefore increased further.

With the example of FIG. 8 there may be provided numerous thermal conductors 30 each of which comprising only a partial cylindrical shape as seen in the circumferential direction. Here, the sidewall 11 of the housing 10 may comprise numerous through openings 16, each of which being filled or provided with an individual thermal conductor 30. Otherwise, and with a cylindrically-shaped thermal conductor 30 as e.g. illustrated in FIG. 4 and with the configuration of FIG. 8, the thermal conductor 30 may be provided between two individual housing components of the injection device 1, e.g. between a proximal housing component 10′ configured to house or to receive the drive mechanism 4 and the protective cap 8 configured to cover the distal end of the injection device 1.

In contrast to that and with the example of FIGS. 6 and 7, the recess 14 having a closed bottom 15 may comprise a circumferential groove that matches in size and shape with the thermal conductor(s) 30 arranged therein.

With the further example of FIG. 9 the thermal conductor 30 is arranged on the inside 12 of the sidewall 11, which may be void of a recess 14. Here, the outside contact surface 34 of the thermal conductor 30 is in direct surface abutment with the rather straight shaped inside 12 of the sidewall 11 of the housing 10.

In FIG. 9 there is illustrated another configuration or example of a mutual arrangement of a medicament container 40′ and a housing 10 of an injection device 1. Here, the medicament container 40′, and in particular the sidewall 41′ of the barrel 42′ is in direct mechanical contact with the inside 12 of the sidewall 11 of the housing 10. For increasing a thermal conductivity of the sidewall 11 of the housing 10, there is embedded a thermal conductor 30′ in the structure of the sidewall 11. Here, the thermal conductor 30′ may comprise a piece of metal or of a comparable heat-conducting material. It is entirely embedded inside the material of the surrounding sidewall 11. It may be insert molded in the sidewall 11. Here, the inside contact surface 32′ and/or the outside contact surface 34′ of the thermal conductor 30 may be covered by the material of the sidewall 11.

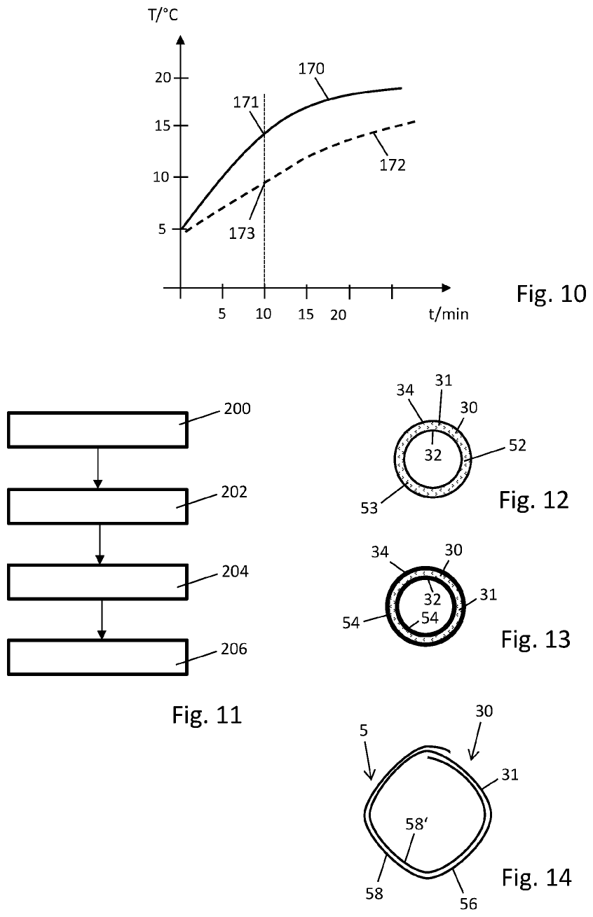

In FIG. 10 there are illustrated to graphs 170, 172 showing an exemplary temporal evolution of a temperature of the medicament container 40 as measured with and without a thermal conductor 30 as described above. The graph 170 reflects the situation with a thermal conductor 30 and the graph 172 represent a situation without the thermal conductor 30.

The graphs 170, 172 show the rise of the container temperature over time right after removing a injection device 1 with the medicament container 40 inside from a refrigerated area at time T=0. As it is immediately apparent from a comparison of the two graphs 170, 172, the injection device 1, which is provided with the thermal conductor 30 provides a faster transfer of thermal energy into the medicament container 40. Here, and after about 10 minutes the medicament container has reached a temperature level 171 close to room temperature. In the situation as reflected by graph 172 and after lapse of the same time interval the temperature level 173 of the medicament container is significantly lower compared to the graph 170.

Using of the thermal conductor 30 on the one hand provides a faster heating and cooling of the medicament 50 when the injection device 1 with the medicament inside is subject to an abrupt change of the environmental temperature. Moreover, with the thermal conductor 30 the measurement precision for the temperature measurement can be significantly increased.

The flowchart of FIG. 11 schematically illustrates a method of measuring the temperature of the medicament 50 located inside the medicament container 40. Here, in a first step 200, the injection device 1 as described herein is provided to a user. In step 202, the injection device 1 is e.g. removed from a refrigerated area and is stored or provided at room temperature. In step 204, the medicament container inside the housing 10 of the injection device 1 becomes subject to a heating due to exposure to the new or raised temperature environment. Since the thermal conductor provides a thermal bridge between the medicament container 40 and the outside surface 18 of the housing 10 respective the exchange of thermal energy between the medicament container 40 and the environment can be accelerated.

In step 206 the thermal conductor 30 is used to conduct a temperature measurement as described above, e.g. in connection with FIG. 2. Optionally, the measurement step 206 is repeated multiple times and the temperature variation, e.g. the heating process of the medicament container can be precisely monitored.

Since the thermal conductivity of the thermal conductor and hence the thermal conductivity of the thermal bridge between the outside surface 18 of the housing 10 and the medicament container 40 is not infinite there may be a certain delay or offset between the actual temperature of the medicament container and the temperature that can be measured at the outside surface 18 of the housing 10 even when using the thermal conductor 30.

In order to compensate for such a temporal delay or temperature offset the measurement software of the external electronic device 150 may be calibrated accordingly. Calibration can be conducted by way of experimental data obtained by a reference measurement of the temperature and temperature development of the medicament 50 inside the medicament container 40 concurrently the with the present measurement as described herein. The reference measurement, e.g. measuring of the actual temperature of the medicament inside the medicament container may be conducted by any suitable contactless temperature measurement, e.g. on the basis of temperature measurement using infrared radiation.

### REFERENCE NUMBERS

1 injection device
2 distal end
3 proximal end
4 drive mechanism
5 piston rod
6 trigger
7 dose dial
8 protective cap
9 window
10 housing
11 sidewall
12 inside
14 recess
15 bottom
16 through opening
18 outside surface
19 marking
20 container receiving space
21 container support
22 container support
30 thermal conductor
31 body
32 inside contact surface
34 outside contact surface
35 distal end
36 slit
38 proximal end
40 medicament container
41 sidewall
42 barrel
43 outlet
44 outside container surface
45 distal end
46 injection needle
47 septum
48 proximal end
49 stopper
50 medicament
52 plastic material
53 dopant
54 coating
56 wire/ribbon
58 winding
100 electronic label
101 substrate
102 foil
104 electric circuit
106 processor
108 antenna
110 temperature sensor
150 external electronic device
152 display
154 temperature indication
158 communication interface
170 graph
171 temperature level
172 graph
173 temperature level

1-18. (canceled)

19. An injection device for injecting a dose of a medicament, the injection device comprising:
a housing, comprising an outside surface and enclosing a container receiving space sized to accommodate a medicament container containing the medicament,
a thermal conductor attached to the housing or integrated into the housing, the thermal conductor comprising an inside contact surface adjoining the container receiving space or extending into the container receiving space to get in direct mechanical contact with the medicament container when the medicament container is arranged in the container receiving space.

20. The injection device according to claim 19, wherein the thermal conductor comprises a heat conducting material with a thermal conductivity larger than 0.5 W/(mK).

21. The injection device according to claim 19, wherein the thermal conductor comprises a heat conducting material with a thermal conductivity larger than 5 W/(mK).

22. The injection device according to claim 19, wherein the thermal conductor comprises a heat conducting material with a thermal conductivity larger than 10 W/(mK).

23. The injection device according to claim 19, wherein the thermal conductor comprises a heat conducting material with a thermal conductivity larger than 200 W/(mK).

24. The injection device according to claim 19, wherein the thermal conductor comprises at least one of a metal, a plastic material with a heat conducting dopant, a polymer with a heat conducting dopant, an elastomeric material with a heat conducting dopant, a glass with a heat conducting dopant, a coiled metal wire, a metal ribbon and a material with a heat conductive coating.

25. The injection device according to claim 19, wherein the housing defines a longitudinal direction (z) and wherein the thermal conductor is compressible or deformable with regard to a radial direction (r) transverse to the longitudinal direction (z).

26. The injection device according to claim 19, wherein the housing comprises a sidewall and wherein the thermal conductor is arranged between the sidewall and the medicament container or between the side wall and the container receiving space.

27. The injection device according to claim 26, wherein the sidewall comprises a recess on an inside and wherein the thermal conductor is arranged in the recess.

28. The injection device according to claim 19, wherein at least a portion of the thermal conductor protrudes inwardly from the inside of the sidewall.

29. The injection device according to claim 19, wherein the inside contact surface of the thermal conductor protrudes inwardly from the inside of the sidewall.

30. The injection device according to claim 29, wherein the recess comprises a through opening extending through the side wall and wherein the thermal conductor extends through the through opening.

31. The injection device according to claim 19, wherein the thermal conductor comprises an outside contact surface flushing with the outside surface of the housing.

32. The injection device according to claim 19, wherein the inside contact surface of the thermal conductor is complementary shaped to an outside surface of the medicament container.

33. The injection device according to claim 19, wherein the inside contact surface of the thermal conductor is of hollow cylindrical shape.

34. The injection device according to claim 19, wherein the thermal conductor comprises a cylindrically shaped body configured to at least partially enclose or to clasp around the medicament container.

35. The injection device according to claim 19, wherein the thermal conductor comprises an annular shaped body or an annular shaped structure having an inside diameter that matches with an outside diameter of a tubular barrel of the medicament container.

36. The injection device according to claim 19, further comprising the medicament container fixed inside the housing and being in direct mechanical and thermal contact with the thermal conductor.

37. The injection device according to claim 19, further comprising a marking on the outside surface, wherein the position of the marking overlaps with a position of the thermal conductor inside the housing.

38. The injection device according to claim 19, further comprising a temperature sensor fastened to an outside of the housing and in thermal contact with the thermal conductor.
